(19) United States
(12) Patent Application Publication
(10) Pub. No.: US 2023/0181703 A1
(43) Pub. Date: Jun. 15, 2023

(54) COMPOSITION COMPRISING PEPSTATIN AND ALGINIC ACID OR A SALT THEREOF, AND USE THEREOF

(71) Applicant: DRUGS MINERALS AND GENERICS ITALIA S.R.L. IN FORMA ABBREVIATA D.M.G. ITALIA S.R.L., Pomezia (RM) (IT)

(72) Inventors: Luigi MERCURI, Pomezia (RM) (IT); Licia TIBERI, Pomezia (RM) (IT)

(21) Appl. No.: 17/925,816

(22) PCT Filed: May 26, 2021
(86) PCT No.: PCT/IB2021/054572

§ 371 Date: Nov. 16, 2022

(30) Foreign Application Priority Data
May 26, 2020 (IT) 102020000012370

(51) Int. Cl. A61K 38/55; A61K 9/00; A61K 9/08; A61K 31/734; A61K 31/728; C07K 7/06

(52) U.S. Cl. CPC A61K 38/55; A61K 9/0048; A61K 9/08; A61K 31/734; A61K 31/728; C07K 7/06; A61K 2300/00

(57) ABSTRACT

The present invention relates to an aqueous composition for ophthalmic use comprising pepstatin and alginic acid or a salt thereof, and to the use of said composition in a method for the treatment of a disease or symptom of the eyeball and/or periocular region related with or deriving from the presence of pepsin in the lacrimal fluid.

The present invention relates to a composition, preferably for ophthalmic use, comprising pepstatin and alginic acid or a salt thereof, and, optionally, a hyaluronic acid or a salt thereof, and to the use of said composition in a method for the treatment of a disease or symptom related with or deriving from the presence of pepsin in extraoesophageal regions, preferably a disease or symptom of the eyeball and/or periocular region related with or deriving from the presence of pepsin in the lacrimal fluid, in subjects in need such as for example subjects with gastroesophageal reflux and/or backflow of gastric fluids from the stomach to an extraoesophageal region (laryngopharyngeal reflux or extraoesophageal reflux).

Gastroesophageal reflux or gastroesophageal reflux disease (in short GERD) is a para-physiological condition characterised by the backflow—from the stomach to the oesophagus—of the gastric content or acidic gastric fluids (pH 1-2) for example comprising pepsin, hydrochloric acid, gastric juices, duodenal juices and acidic gastric fluids. Thus, in patients with GERD, the backflow—even in very small amounts—of the gastric content from the stomach to the oesophagus causes the irritation of the epithelium of the oesophagus, which causes a burning sensation at the retrosternal position and pains upon deglutition, as well as an increase in caries (due to the corrosion of the teeth enamel caused by the gastric acids), retching after taking food, feeling of acidity in the upper part of the oesophagus and in the pharynx.

Gastroesophageal reflux is a very common diseases in the population, often related with obesity, diabetes mellitus, increased gastric secretion conditions, pregnancy, smoke, alcohol, hiatus hernia.

In the absence of an endoscopic damage visible under gastroscopy, gastric reflux is defined as non-erosive reflux or non-erosive reflux disease (in short NERD) given that the reflux of the acid content does not cause esophagitis.

Laryngopharyngeal reflux (in short LPR) or laryngopharyngeal reflux disease or, alternatively, extraoesophageal reflux are the various terms used to identify the symptoms or disorders or diseases caused by the backflow of the gastric content from the stomach to the extraoesophageal regions. Said extraoesophageal regions may be the upper respiratory tract, the eyeball (or eye), the periocular region (e.g. eyelid, conjunctiva and lacrimal apparatus), and the ear apparatus, the Eustachian tube and the middle ear.

In the context of the present invention, the periocular region is defined as the region around the eye or regarding the eye contour, such as for example the eyelid, the conjunctiva and the lacrimal apparatus. The eyelid is a skin-membrane formation that covers the eye. The conjunctiva is a mucosa membrane, which covers the eyeball and the inner part of the eyelids. The lacrimal apparatus is the set of secretory system of the tear film, which includes the lacrimal gland, and of the apparatus that allows the outflow thereof. The apparatus that allows the outflow thereof includes the lacrimal ways, consisting of: lacrimal points, lacrimal sac and lacrimal channels or lacrimal ducts or nasolacrimal duct.

When the gastric content or part thereof (e.g. pepsin) backflows from the stomach, at the level of the extraoesophageal regions there occur inflammatory and/or irritation phenomena which can involve one or more of said extraoesophageal regions, for example the upper respiratory tract, oral cavity, ear cavity, eyeball (or eye) and/or periocular region (e.g. eyelid, conjunctiva and lacrimal apparatus).

The two diseases, GERD and LPR or extraoesophageal reflux, can coexist or manifest themselves separately.

In the case where GERD and LPR coexist, besides disorders in the oesophageal region, patients with GERD may also suffer from disorders or diseases in one or more extraoesophageal regions.

Should GERD and LPR not coexist, disorders or symptoms felt in one or more extraoesophageal regions may also occur in subjects not with GERD, following the backflow of the gastric content from the stomach to the extraoesophageal area.

In light of the above, people with LPR (or erosive or non-erosive) or LPR (or extraoesophageal reflux) can suffer, for example, from the lacrimal dysfunction syndrome (in short LDS, or ocular surface disease (OSD) alternatively defined as the dry eye syndrome. As a matter of fact, the presence of pepsin (component of the gastric content) was detected in the lacrimal secretion of the subjects with gastroesophageal reflux.

The lacrimal dysfunction syndrome (LDS) is an eye disease which consists in a quantity reduction and/or in a quality alteration of the tear film, which mainly has a function of moistening and protecting the front surface of the eyeball. The tear film has the task of performing the following crucial functions:

i) nutritional: tears allow to ensure a correct supply of oxygen and nutrients for an appropriate turnover of the cells of the ocular surface;

ii) antimicrobial: the presence of antibodies and enzymes in the tear film ensures a defensive action against external aggressions;

iii) cleaning; and

iv) lubricating.

Thus, the change in the quantity/quality of the tear film entails exposing the eyeball to greater friction (determined by the movement of the eyelids) and to a greater risk of infections. The lacrimal dysfunction syndrome is among the most frequent diseases in ophthalmology and the impact thereof is high in the middle age and elderly population.

In the context of the present invention, the expressions “lacrimal dysfunction syndrome” (LDS) and “dry eye syndrome” will be used interchangeably.

Another disease or symptom that may occur in subjects with GERD, LPR or extraoesophageal reflux is the inflammation of the eyeball or the inflammation of the periocular region, in particular conjunctivitis, that is an inflammation that affects the conjunctiva causing the reddening of the eyes and conferring the red eyes characteristic. Conjunctivitis may affect adults, children and newborns alike.

Frequently, disorders or symptoms or diseases in extraoesophageal regions, such as the eyeball and the periocular region, are analysed, diagnosed and/or treated without considering a possible correlation with the phenomenon of the backflow of the gastric content, for example pepsin, from the stomach to the extraoesophageal regions.

As a result, many of said diseases or symptoms of the eyeball and/or periocular region, such as for example the lacrimal dysfunction syndrome (SDL), conjunctivitis or ocular inflammation or periocular inflammation are treated with products currently available on the market (for example steroidal products) which do not specifically treat the cause that triggers said diseases or symptoms, but only provides a temporary remedy; for example said products do not effectively treat the presence of pepsin in the lacrimal fluid, eyeball and/or periocular region.

BALESTRAZZI ALESSANDRA ET AL: “A new therapeutic approach for the Dry Eye Syndrome in patients with laryngopharyngeal reflux: first data” is a preliminary study of comparison between two treatments in patients with the dry eye syndrome and LPR. The patients were treated with Gastroftal eye drops and Gastroftal tablets, or with hyaluronic acid eye drops (Atlantis) alone for 3 months. Gastroftal eye drops does not contain pepstatin.

WO 2008/039984 A2 describes compounds which are inhibitors of cathepsin D—a member of the subfamily of aspartyl protease—and pharmaceutical compositions which contain said inhibitors. Such compounds have neurotrophic activity, and they are useful in the treatment and in the prevention of neuronal disorders such as amyotrophic lateral sclerosis, multiple sclerosis and muscular dystrophy. Said pharmaceutical compositions contain alginic acid or a salt thereof.

U.S. Pat. No. 4,339,439 A shows a greater anti-ulcers activity obtained in warm-blooded animals with a concomitant administration of etintidine, an antagonist of the histamine H2 receptor, and of pepstatin, a pepsin complexing agent.

The technical problem addressed and solved by the present invention lies in providing compositions or mixtures for ophthalmic use or for nasal use or for use in the oral cavity in methods for the preventive and/or curative treatment of diseases or symptoms in extraesophageal regions, preferably diseases or symptoms of the eyeball and/or periocular region such as for example lacrimal dysfunction syndrome (LDS), conjunctivitis, ocular inflammation or periocular inflammation, related with or deriving from the presence of pepsin in said extraesophageal regions, preferably in the lacrimal fluid, primarily due to a gastric reflux in the esophageal and/or extraesophageal region.

Furthermore, the technical problem addressed and solved by the present invention lies in providing compositions or mixtures for ophthalmic or nasal use or in the oral cavity in said treatment methods that are stable, effective, easy to administer/apply, well-tolerated and basically free of side effects.

Following an extensive and in-depth research and development activity, the Applicant addresses and solves the aforementioned technical problems by providing compositions or mixtures (in short, compositions or mixtures of the invention) for ophthalmic use or for nasal use or for use in the oral cavity comprising pepstatin and alginic acid or a salt thereof and, optionally, hyaluronic acid or a salt thereof as described hereinafter. Advantageously, the compositions and mixtures of the invention are free of steroidal substances.

Besides being effective in the treatment of extraoesophageal disorders, preferably of the cited inflammatory ocular and/or periocular disorders, the compositions or mixtures of the invention for ophthalmic use based on pepstatin and alginic acid or a salt thereof and, optionally, hyaluronic acid or a salt thereof are stable, easy to administer/apply, well-tolerated and basically free of side effects.

In particular, the combination of pepstatin and alginic acid or a salt thereof acts effectively and synergistically to reduce and eliminate pepsin from the lacrimal fluid. This effectiveness is primarily due to the inhibitory action of pepstatin, even at low concentrations, and to the simultaneous action of alginate both as sequestrant of pepsin through a non-specific binding and as a lubricant.

Furthermore, the addition of an optional lubricating agent preferably hyaluronic acid or a salt thereof, confers viscoelastic activity to the composition of the invention.

Furthermore, the ophthalmic compositions or mixtures of the invention preferably do not contain steroidal substances.

Lastly, the compositions for ophthalmic use of the present invention are easy to prepare and cost-effective.

These and other objects which will be apparent from the detailed description that follows, are attained by the mixtures and by the composition of the present invention due to the technical characteristics claimed in the attached claims.

### FIGURES

FIG. 1: absorption spectra of the blank and of the sample (pepsin) without the presence of the inhibitory substance (pepstatin); conditions: pepsin 0.02%, haemoglobin 2%, pH 1.5, 37° C.

FIG. 2: absorption spectra of the blank and of the pepsin sample in the presence of pepstatin (pepstatin: suspension in H₂O) and comparison with the spectra relating to free pepsin without the presence of pepstatin.

FIG. 3: absorption spectra of the blank (pepsin without pepstatin) and of the sample (pepsin+pepstatin) (pepstatin: dissolved in EtOH and diluted in water).

FIG. 4: pepsin activity spectra in the presence of an aqueous solution comprising magnesium alginate; conditions: pepsin 0.02%, haemoglobin 2%, 1% of aqueous solution comprising magnesium alginate; pH 1.5, 37° C.

FIG. 5: absorption spectra of the blank (pepsin in the absence of pepstatin but in the presence of an aqueous solution comprising magnesium alginate) and of the pepsin samples in the presence of an aqueous solution comprising magnesium alginate and pepstatin (pepstatin diluted in alginate aqueous solution after dissolution in EtOH).

### DETAILED DESCRIPTION OF THE INVENTION

Forming an object of the present invention is a composition comprising (i) a mixture M of active components comprising or, alternatively, consisting of:

(a) pepstatin or a pharmaceutically acceptable salt thereof, preferably an alkali metal or alkaline earth metal salt (e.g. magnesium, sodium, potassium or calcium), and

(b) an alginic acid or a pharmaceutically acceptable salt thereof, preferably an alkali metal or alkaline-earth metal alginate (e.g. magnesium, sodium, potassium or calcium);

and, optionally, said composition comprises (ii) at least one pharmaceutical or ophthalmological grade additive and/or excipient.

Pepstatin (alternatively referred to as pepstatin A) is an aspartyl protease inhibitor. It is a hexapeptide containing the amino acid statin (Sta, (3S,4S)-4-amino-3-hydroxy-6-methylheptanoic) acid, with the sequence lsovaleryl-Val-Val-Sta-Ala-Sta (Iva-Val-Val-Sta-Ala-Sta) (example of CAS No.: 26305-03-3). Pepstatin is a molecule produced by actinomycete bacteria. At neutral pH (buffered) pepstatin is in acidic form and it can be dissolved in alcohol (for example ethanol) and then diluted in water. When in salt form instead, pepstatin can be dissolved in water at neutral pH. Preferably, for the preparation of the mixtures or compositions of the present invention, comprising (a), (b) and optionally (c) and/or other components described in the present invention, pepstatin is used as such, that is pepstatin not in the form of salt.

Together with (a) and, optionally, (c) and/or other components described in the present invention, the alginic acid comprised in the mixture or composition of the invention is preferably an alginic acid (for example CAS No. 9005-32-7) having an average molecular weight comprised in the range from about 50 kDalton (kDa) to about 800 kDa; preferably from about 100 kDa to about 600 kDa; more preferably from about 200 kDa to about 400 kDa, for example to about 240 kDa (atomic mass units). Advantageously, said (b) alginic acid or a salt thereof, is obtained from marine algae.

In a preferred embodiment, the mixture M of active components, comprised in the composition of the invention, comprises or, alternatively, consists of: (a) pepstatin or a salt thereof, (b) an alginic acid, or a salt thereof, preferably an alkali metal or alkaline earth metal salt (e.g. magnesium, sodium, potassium or calcium), more preferably magnesium alginate, and (c) a lubricating agent, preferably hyaluronic acid or a pharmaceutically acceptable salt thereof, more preferably an alkali metal or alkaline earth metal hyaluronate (e.g. magnesium, sodium, potassium or calcium),even more preferably sodium hyaluronate.

Together with (a) and (b) according to any one of the embodiments of the present invention, said lubricating agent (c), optionally comprised in the mixture M of the composition of the invention may be selected from: hyaluronic acid or a salt thereof, carboxymethyl cellulose, hypromellose, xanthan gum, polyvinylpyrrolidone (PVP), polyvinyl alcohol, and mixtures thereof; preferably hyaluronic acid or a salt thereof.

Together with (a) and (b) and optionally other components described in the present invention, hyaluronic acid or a salt thereof (e.g. sodium hyaluronate) comprised in the mixture or composition of the invention preferably is or is derived from a linear or branched hyaluronic acid with an average molecular weight comprised in the range from about 200 kDa to about 5,000 kDa; preferably from about 1,000 kDa to about 3,000 kDa; more preferably from about 1,500 kDa to about 2.000 kDa.

According to a preferred example, the composition of the invention comprises or, alternatively, consists of: (a) pepstatin; (b) alkali metal or alkaline earth metal alginate, preferably magnesium alginate, (c) alkali metal or alkaline earth metal hyaluronate, preferably sodium hyaluronate and ophthalmological grade additives and/or excipients.

According to a more preferred example, the composition of the invention comprises or, alternatively, consists of: (a) pepstatin, (b) magnesium alginate, (c) sodium hyaluronate, and ophthalmological grade additives and/or excipients.

According to a further preferred example, the composition of the invention comprises or, alternatively, consists of: (a) pepstatin, (b) magnesium alginate from alginic acid obtained from marine algae having an average molecular weight from about 200 kDa to about 400 kDa, for example to about 240 kDa, (c) sodium hyaluronate with an average molecular weight from about 1,500 kDa to about 2,000 kDa, and ophthalmological grade additives and/or excipients.

Besides (a) pepstatin, (b) alginic acid or a salt thereof, and, optionally, (c) a lubricating agent, preferably hyaluronic acid or a salt thereof, the mixture M comprised together with the additives in the composition of the invention may additionally comprise at least one further active component (d) selected from the group consisting of: plant extracts (for example extracts of green tea (Camellia sinensis), amino acids, vitamins of group A, B, C, D and/or E, and mixtures thereof.

The compositions of the present invention, comprising said mixture M comprising (a), (b), and, optionally (c), may comprise at least one acceptable pharmaceutical or food grade additive and/or excipient, that is a substance devoid of therapeutic activity suitable for pharmaceutical or food use. In the context of the present invention, the acceptable additives and/or excipients for pharmaceutical or food use comprise all the ancillary substances known to the person skilled in the art for the preparation of compositions in semi-solid or liquid form, such as, for example, diluents, solvents (including water), solubilisers, acidifiers, thickeners, sweeteners, lubricants, surfactants, preservatives, stabilisers, pH stabilising buffers and mixtures thereof.

The composition and the mixture M of the present invention, comprises (a), (b), and optionally (c) according to any one of the described embodiments, preferably do not comprise a steroidal compound.

According to an aspect of the invention, the mixtures or compositions of the invention are not formulated for oral use.

The composition for ophthalmic use of the invention, comprising (a), (b) and, optionally, (c) (according to any one of the described embodiments) may be in liquid form, such as water-based eye drops, and it shows high stability (preferably≥(greater than or equal to) 24 months), homogeneity and no formation of precipitates in liquid form, preferably water or oil-based eye drops, or in semi-solid form, preferably an ointment, a salve, a gel or a cream.

The composition of the invention in liquid form, for example in the form of eye drops, may comprise pepstatin in a molar concentration (M) comprised in a range from 0.1 nM (0.0001 μM) to 50 μM; preferably from 0.01 μM to 5 μM; more preferably from 0.5 μM to 2 μM (for example about 1 μM).

According to an aspect of the invention, the composition of the invention may comprise, in percentage (%) by weight with respect to the total weight of the composition: (a) pepstatin as such from 0.0000001% (1×10⁻⁸%) a 0.001% (1×10⁻³%), preferably from 0.000001% (1×10⁻⁶%) to 0.0003% (3×10⁻⁴%), more preferably from 0.00003% (3×10⁻⁵%) to 0.0001% (1×10⁻⁴%), (b) alginic acid or a salt thereof, preferably magnesium alginate, from 0.01% to 10%, preferably from 0.05% to 2%, more preferably from 0.05% to 0,4%, for example 0,2%; and, optionally, (c) lubricating agent, preferably hyaluronic acid or a salt thereof, more preferably sodium hyaluronate, from 0.01% to 10%; preferably from 0.05% to 2%; more preferably from 0.05% to 0.30%, for example 0.15%.

In the context of the present invention, the expression “composition” is used to indicate a pharmaceutical composition or medical device composition according to the European regulation on medical devices [EU 2017/745 —(MDR), Directive 93/42/EEC—(MDD)].

Forming an object of the present invention are the compositions and the mixture M of the present invention, comprising (a), (b), and optionally (c) and/or additives/excipients according to any one of the described embodiments, for use as medicament.

Forming an object of the present invention are the compositions and the mixture M of the present invention, comprising (a), (b), and optionally (c) and/or additives/excipients according to any one of the described embodiments, for use in a method for preventive and/or curative treatment of a disorder or disease of the eyeball and/or periocular region, preferably of inflammatory nature, such as for example lacrimal dysfunction syndrome (LDS), conjunctivitis, ocular inflammation or periocular inflammation (for example blepharitis, keratitis, uveitis), primarily related with or deriving from the presence of pepsin in the lacrimal fluid, in subjects in need.

The presence of pepsin in the lacrimal fluid is plausibly due to a gastric reflux in the esophageal and/or extraesophageal region.

The presence of pepsin in the lacrimal fluid may be determined through conventional diagnostic methods and methods known to the person skilled in the art suitable to detect pepsin in a liquid.

The presence of pepsin in the lacrimal fluid manifests itself in subjects with a gastric reflux and/or a disease or symptom associated with said gastric reflux.

Said diseases or symptoms associated with gastric reflux are selected from the group comprising or, alternatively, consisting of: gastroesophageal reflux disease (GERD), laryngopharyngeal reflux disease (RFL) or extraoesophageal reflux, esophagitis (acute or chronic inflammation of the oesophagus mucosa), oesophageal ulcers, oesophageal mucosal de-epithelialisation, acid regurgitation, heartburn, feeling of gastric fullness, epigastric pain, dyspepsia, nausea, chronic cough, bronchospasm, sore throat, laryngitis, globus sensation or hypopharyngeal bolus, pyrosis, dysphonia, rhinopharyngeal phlogosis, and all disorders primarily caused or co-caused by gastric reflux.

Said diseases or symptoms associated with gastric reflux, including the disease or symptom of the eyeball and/or periocular region, preferably of inflammatory nature, such as for example lacrimal dysfunction syndrome (SDL), conjunctivitis, ocular inflammation or periocular inflammation (for example blepharitis, keratitis, uveitis), may be present even in the absence of a diagnosis of gastroesophageal reflux disease (GERD), that is in subjects not suffering from GERD.

Furthermore, the present invention further relates to a process for preparing the mixture or composition of the invention (in short, process of the invention), preferably for ophthalmic use, comprising a step 1) for dissolving pepstatin in a suitable solvent (for example alcoholic solvent, ethanol or ethyl alcohol, isopropyl alcohol, glycerine, propylene glycol, sorbitol 20 or Tween® 20 (mixture of partial tri-esters of sorbitol with the mono and di-anhydrides thereof with stearic acid), ethoxylated hydrogenated castor oil 40 moles (Peg-40 hydrogenated castor oil); preferably in an alcoholic solvent, more preferably ethanol) at neutral pH (obtained using a buffer solution; preferably a borate buffer solution) to obtain a pepstatin alcoholic solution, preferably a pepstatin alcoholic solution at a concentration comprised in the range from 0.01 mg/ml to 1 mg/ml, preferably comprised from 0.1 mg/ml to 1 mg/ml, for example 1 mg/ml. Said step 1) is followed by the step 2) for diluting the pepstatin alcoholic solution in an aqueous solution comprising an alginic acid salt, preferably an alkali metal or alkaline earth metal alginate, more preferably magnesium alginate.

Pepstatin at neutral pH is not soluble in water and—in the prior art—pepstatin is used in the form of aqueous suspension.

On the contrary, the process of the invention allows to dissolve pepstatin in aqueous phase and without using solvents that are potentially toxic or unsuitable for ophthalmic use.

For the sake of clarity, in order to achieve the object of the present invention, the components (or active components) (a), (b) and, optionally (c) of the mixture or composition of the invention can be administered simultaneously or separately, (preferably in a time interval from 5 minutes to 30 minutes) and in any order; preferably, the active components are administered to a subject simultaneously, even more preferably in a single composition to obtain a more rapid effect and for ease of administration. When the active components of the invention are administered in a single composition, said single composition corresponds to the composition of the present invention.

In the context of the present invention and the expression “subject/s” is used to indicate human or animal subjects, preferably mammals (e.g. pets such as dogs, cats, horses, sheep or bovines). Preferably, the compositions of the invention are for use in methods for the treatment of human subjects.

Unless specified otherwise, the expression composition or mixture or other comprising a component at an amount “comprised in a range from x to y” is used to indicate that said component may be present in the composition or mixture or other at all the amounts present in said range, even though not specified, extremes of the range included.

Unless specified otherwise, the content of a component in a composition or mixture refers to the percentage (%) by weight of that component with respect to of said total weight of said composition or mixture.

Unless specified otherwise, the indication that a composition or mixture or other “comprises” one or more components means that other components can be present besides that or those specifically indicated, and the indication that a composition or mixture or other “consists” of determined components means that the presence of other components not indicated is excluded.

In the context of the present invention, the expression “treatment method” is used to indicate an intervention on a subject in need, comprising the administration of a composition or mixture of the invention to the subject at a therapeutically effective amount, with the aim of eliminating, reducing/decreasing or preventing a disease or ailment and the symptoms or disorders thereof.

The expression “therapeutically effective amount” refers to the amount of active compound that elicits the biological or medicinal response in a tissue, system, mammal, or human being that is sought and defined by an individual, researcher, veterinarian, physician, or other clinician or health worker.

### EXAMPLES

Examples of ophthalmic composition in liquid form according to the invention are reported in Table 1, 2, 3 and 4 (solutions buffered to about pH 7.6±0.1: 7.4-7.8±0.1).

TABLE 1

| Component | % weight/total weight (w/w) |
|---|---|
| Purified water | from 90% to 99% |
| Pepstatin or | 1 × 10⁻⁸ to 1 × 10⁻³ (3 × 10⁻⁵%-1 × 10⁻⁴%) |
| Pepstatin in EtOH 1 mg/ml | from 0.01% to 1% (0.01%-0.1%) |
| Magnesium alginate | from 0.01% to 10% (0.05%-0.4%) |
| Additives and/or excipients | q.s at 100 |

TABLE 2

| Component | % weight/total weight (w/w) |
|---|---|
| Purified water | from 90% to 99% |
| Pepstatin or | 1 × 10⁻⁸ to 1 × 10⁻³ (3 × 10⁻⁵%-1 × 10⁻⁴%) |
| Pepstatin in EtOH 1 mg/ml | from 0.01% to 1% (0.01%-0.1%) |
| Magnesium alginate | from 0.01% to 10% (0.05%-0.4%) |
| Di-sodium tetraborate | from 0.01% to 5% (0.05%-0.50%) |
| Boric acid | from 0.01% to 5% (0.30%-1.50%) |
| Sodium chloride | from 0.01% to 5% (0.10%-1.00%) |
| optional, other additives | q.s at 100 |
| and/or excipients | |

TABLE 3

| Component | % weight/total weight (w/w) |
|---|---|
| Purified water | from 95% to 99% |
| Pepstatin or | 1 × 10⁻⁸ to 1 × 10⁻³ (3 × 10⁻⁵%-1 × 10⁻⁴%) |
| Pepstatin in EtOH 1 mg/ml | from 0.01% to 1% (0.01%-0.1%) |
| Magnesium alginate | from 0.01% to 10% (0.05%-0.4%) |
| Hyaluronic acid sodium salt | from 0.01% to 10% (0.05%-0.30%) |
| Additives and/or excipients | q.s at 100 |

TABLE 4

| Component | % weight/total weight (w/w) |
|---|---|
| Purified water | from 95% to 99% |
| Pepstatin or | 1 × 10⁻⁸ to 1 × 10⁻³ (3 × 10⁻⁵%-1 × 10⁻⁴%) |
| Pepstatin in EtOH 1 mg/ml | from 0.01% to 1% (0.01%-0.1%) |
| Magnesium alginate | from 0.01% to 10% (0.05%-0.4%) |
| Hyaluronic acid sodium salt | from 0.01% to 10% (0.05%-0.30%) |
| Di-sodium tetraborate | from 0.01% to 5% (0.05%-0.50%) |
| Boric acid | from 0.01% to 5% (0.30%-1.50%) |
| Sodium chloride | from 0.01% to 5% (0.10%-1.00%) |
| optional, other additives | q.s at 100 |
| and/or excipients | |

### Experimental Part

### I. Method

The method used to measure the enzymatic activity of pepsin alone or together with the inhibitory substance (pepstatin), consists of an adaptation of the Anson method [Anson et al, J. Gen. Physiol. 1931, 16, 59] which provides for the use of denatured haemoglobin as substrate. The enzymatic activity was determined after precipitation with trichloroacetic acid of the non-hydrolysed substrate. The activity of the enzyme was determined spectrophotometrically from the concentration of soluble peptides released by the proteolytic action.

Reaction studied:

(Haemoglobin+H₂O)+pepsin→oligopeptides

### II. Reagents:

Hydrochloric acid 12 M.

Humans haemoglobin (Sigma-Aldrich).

Pepsin (BDH; 1 Anson units per gram): enzyme.

Trichloroacetic acid solution 6.1 N (Sigma-Aldrich).

Pepstatin A: inhibitory substance (or inhibitor).

### III. Conditions

T=37° C., pH=1,5, Aₙₘ, optical path 1 cm, reaction time 10 minutes. At pH=1.5, in which the experiments were conducted, there is a maximum activity of pepsin.

Haemoglobin 2%, pepsin 0,02%, inhibitor 0.01%.

### IV. Preparation

Substrate: 250 mg of human haemoglobin are weighed in a 10 mL flask, it is brought up to volume with distilled water and left under stirring for 10 minutes in a water bath at 37° C. to obtain a solution 2.5%. The solution is passed through a filter paper to eliminate insoluble residues. 8 mL of the filtrate are taken and the volume is brought up to 10 mL with acidic water so as to have a final pH finale of 1.5 and a solution 2% (w/V).

Enzyme: 5 mg of pepsin are weighed in a 5 mL flask and it is brought up to volume with acidic water (pH=1.5).

Enzyme together with inhibitory substance: 5 mg of pepsin are weighed in a 5 mL flask. 0.5 mg/mL of a solution or suspension at pH=1.5 of the inhibitory substance are prepared separately (checking the pH with a pH meter and correcting it, if necessary, with hydrochloric acid 12 M in an amount such not to change the total volume). The flask containing pepsin is brought up to volume with the solution or suspension of the inhibitory substance 0.5 mg/mL and the entirety is kept under magnetic stirring for 10 minutes prima before prior to beginning the experiment.

Trichloroacetic acid: a trichloroacetic acid solution 6.1 N is diluted 20 folds in distilled water to obtain a solution 5% (wN).

### V. Experimental Procedure

0.25 mL of substrate (haemoglobin) are pipetted in a suitable vial and almost entirely submerged in a water bath thermostated at 37° C. It is left under magnetic stirring for 10 minutes at 37° C. 50 microlitres of the enzyme solution (pepsin) or of the enzyme together with the inhibitory substance (pepsin+pepstatin A) are then added and the system is kept under incubation for exactly 10 minutes. 0.5 mL of the trichloroacetic acid solution 5% are added after this period of time and then left for another 5 minutes. Exactly 3 mL of distilled water at pH=1.5 are added into the vial using a graduated pipette; the solution is stirred and filtered using a 0.45 μm syringe filter. An absorption spectrum (with absorbance (A) always below 1) with wavelengths comprised between 300 nm and 250 nm is recorded using a spectrophotometer using the absorbance value at 280 nm as reference.

Blank: 0.25 mL of substrate (haemoglobin) are pipetted in a suitable vial and almost entirely submerged in a water bath thermostated at 37° C. It is left under magnetic stirring for 10 minutes. 50 microlitres 0.5 mg/mL of the inhibitory substance solution (without enzyme) are then added and the system is kept under incubation for exactly 10 minutes. 0.5 mL of the trichloroacetic acid solution 5% are added after this period of time and then left for another 5 minutes.

50 microlitres of solution 1 mg/mL of enzyme without inhibitory substance are the introduced. Exactly 3 mL of distilled water at pH=1.5 are added into the vial using a graduated pipette, then the solution is stirred and filtered using a 0.45 μm syringe filter. An absorbance with wavelengths comprised between 300 nm and 250 nm is recorded using a spectrophotometer using the absorbance value at 280 nm as reference.

### VI. Calculations

Each experiment was repeated at least twice. The final absorbance value (A) at 280 nm was the mean of the absorbance values obtained (with an uncertainty on the values always much lower than 10%). A ΔA, with ΔAₓ=A_{280sample substanceX}−A_{280blank substance X} and ΔAₒ=A_{280sample free pepsin}−A_{280blank free pepsin} in the event of absence of the inhibitory substance are associated with the pepsin-inhibiting substance. The ΔA found is proportional to the amount of soluble aromatic amino acid residues released in the solution due to the proteolytic action of pepsin alone or in the presence of the inhibitor (pepstatin A).

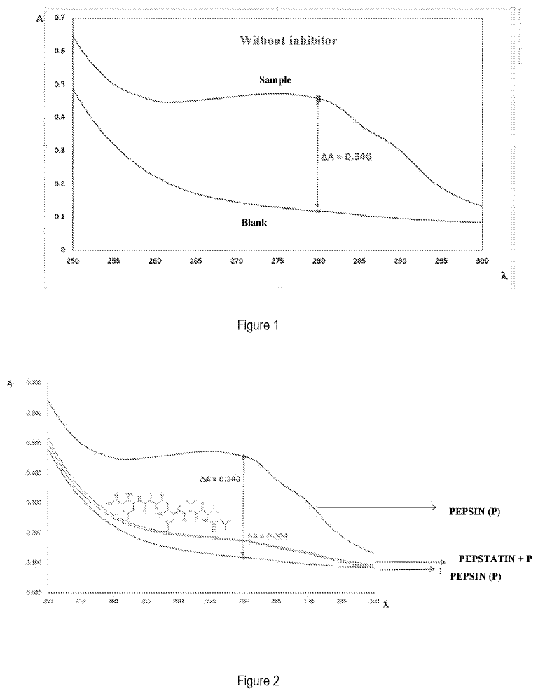

FIG. 1 reports the absorption spectra of the blank and of the sample (reaction time=10 minutes) without the presence of any inhibitor. ΔAₒ=0.40.

The maximum absorbance difference between the sample and the blank at 280 nm found is equal to 0.34 and it regards the proteolytic action of the pepsin enzyme against the substrate within a 10-minute period of time without the presence of any inhibitor under these experimental conditions.

The residual activity in the presence of substance X was calculated based on the equation below:

Residual activity=(ΔAₓ/66 Aₒ)×100 Equation 1

### VII. Results and Discussion

### VII.A. Pepstatin

TABLE 5
Values of ΔA_{X} at 280 nm regarding the substance studied and of
the residual activity with respect to the maximum activity measured.

| Substance | ΔA_{X} | Residual activity |
|---|---|---|
| None | 0.340 | 100% |
| PEPSTATIN | 0 | 0% |

The concentration of the substance (0.01%) was selected both so as to observe an inhibition against pepsin and so as to reduce the optical diffusion caused by the inhibitor to the maximum. The Anson method used allows to normalise the increase in absorbance regarding diffusion which has an equivalent effect both on the blank and on the sample for each substance. As a matter of fact, an absorbance difference at 280 nm was measured irrespective of the diffusion phenomena.

Pepstatin showed a total abatement in the residual activity (ΔAₓ=0).

Pepstatin is insoluble in H₂O, but the suspension produced in laboratory was fully effective for the purposes sought (ΔAₓ=0).

FIG. 2 reports the absorption spectra of the blank and of the sample (reaction time=10 minutes) in the presence of pepstatin 0.01% (suspension in H₂O; greater absorbance of the blank due to the pepstatin suspension), ΔAₒ=0.004, and comparison with the spectra regarding free pepsin without the presence of inhibitors ΔAₓ=0.340.

Pepstatin was perfectly soluble in ethanol (EtOH) at a concentration of 1 mg/mL. Such solution can be diluted in water. Pepstatin maintains the inhibitory activity thereof even after diluting in water from EtOH and it remains soluble; this is proven by the fact that it maintains the inhibitory ability thereof and by the fact that no scattering effect is observed with respect to the blank (aggregation index of the molecules not found in this case).

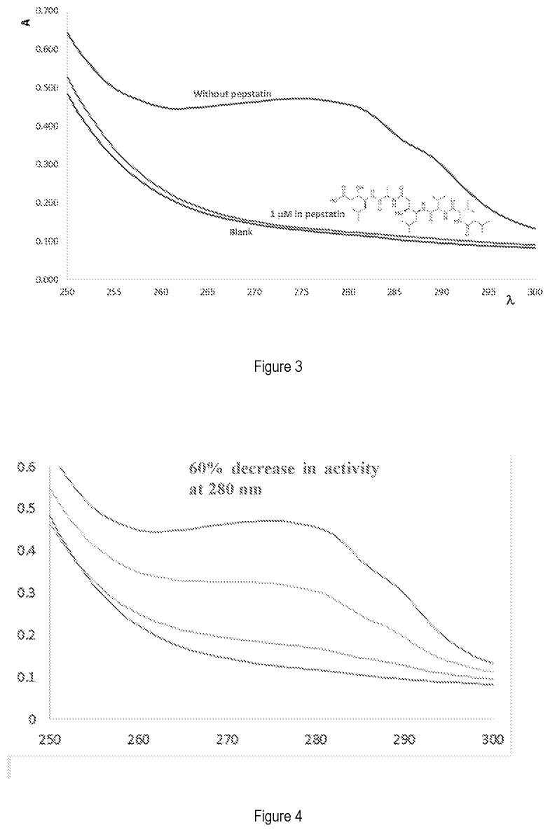

The activity of pepsin in the presence of pepstatin 1 μM was then measured under test conditions identical to the previous tests. Pepstatin was added to haemoglobin 2% before adding pepsin. In the light of the above, it was studied whether the binding of the enzyme with the inhibitor was immediate or slower in the time scale of the experiment (t=10 minutes) (kinetics study). Haemoglobin solutions 2%, 1 μM in pepstatin were prepared and the various activities were measured after adding the enzyme. The results the experiment are shown in FIG. 3. FIG. 3 reports the absorption spectra of the blank (pepsin without pepstatin) and of the sample (reaction time=10 min) at pH=1.5 in the presence of pepstatin (diluted in H₂O after dissolution in EtOH 1 mg/mL) 1 μM.

FIG. 3 immediately shows a total abatement in the activity at the concentration of 1 μM. From a kinetic point of view, given that the activity is basically null, it is clear that this is an immediate binding between the enzyme and the inhibitor given that the enzyme does not have the time to perform the proteolytic action thereof when the inhibitor is already present in the haemoglobin 2%.

### VII.B. Magnesium Alginate

A sterile aqueous solution containing magnesium alginate 0.2% w/w (in short, alginate solution) was added to the haemoglobin solution as a subsequent step of the experiments.

The selected concentration of said alginate solution in haemoglobin was 1%. This concentration allows to avoid scattering and not have a total abatement of the activity. The alginate solution in haemoglobin showed to inhibit pepsin with a 60% decrease in activity with respect to free pepsin at pH=1.5 (FIG. 4). FIG. 4 reports the decrease in activity of pepsin in the presence of magnesium alginate aqueous solution 1% at pH=1.5.

### VII.C. Pepetatin+Magnesium Alginate

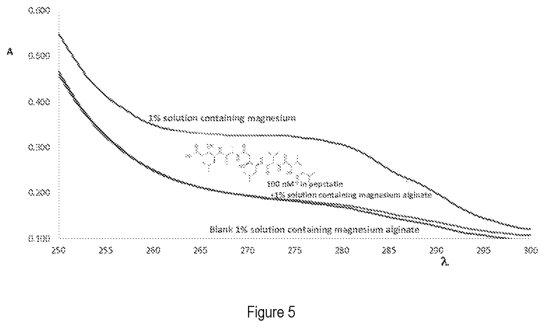

In order to test a synergistic effect of pepstatin together with the magnesium alginate (compositions according to the invention) in totally inhibiting pepsin, solutions at pH=1.5 of haemoglobin in which there was present 1% of said aqueous solution containing magnesium alginate 0.2% w/w (see paragraph VII.B.) and 1 μM in pepstatin dissolved in such solution (in the amount of solution containing alginate added to haemoglobin there was an amount of pepstatin such to obtain the final concentration in pepstatin sought) were prepared and the various activities were measured after adding the enzyme (pepsin). The results the experiment are shown in FIG. 5.

The dilution of a ethanolic solution of pepstatin 1 mg/mL in the magnesium alginate aqueous solution does not jeopardise the dissolution and the inhibitory ability of pepstatin.

FIG. 5 reports the absorption spectra of the blank (pepsin without pepstatin but with 1% in haemoglobin of solution containing magnesium alginate) and of the samplesi (reaction time=10 min) at pH=1.5 in the presence of 1% of magnesium alginate solution and pepstatin 100 nM (pepstatin diluted in magnesium alginate aqueous solution after dissolution in EtOH 1mg/mL).

The synergistic effect of pepstatina together with magnesium alginate allowed to obtain a total abatement of the pepsin activity through a concentration in pepstatin equal to100 nM (an order of magnitude less with respect to pepstatin in water).

### VIII. Conclusions

Pepsin may be present in the tears in patients with gastroesophageal reflux. Such enzyme causes damage to the eye due to the proteolytic action when it is still active. Alginates inhibit pepsin in a non-competitive and dose-dependent manner. Pepstatin A instead, inhibits pepsin in picomolar concentrations. The pepstatin-magnesium alginate binary system has a synergistic inhibitory effect which totally deactivates the pepsin enzyme right from the picomolar concentrations due to pepstatin, and it sequestrates such enzyme through a non-specific binding by the magnesium alginate which at the same time carries out a lubricating action. Such binary system therefore cures and prevents the ocular damage caused by pepsin.

Pepstatin is not soluble in water; as a matter of fact, it has always been used as a suspension capable of inhibiting pepsin in any case. The Applicant found a method through which such inhibitor could be dissolved in water without using toxic solvents. An ethanolic solution of pepstatin can be diluted in water. It still maintains its inhibitory activity and remains soluble after dilution. The pepstatin dissolved in water through this method is able to inhibit pepsin stoichiometrically. There was no presence of suspensions or precipitates in solution. Binding with pepsin in water is immediate in the human time scale to the extent that the pepsin added to haemoglobin during the experiments cannot carry out the proteolytic action thereof when the inhibitor is present.

### IX. Preparation Method

Described hereinafter are the steps for the preparation of a composition in aqueous-based liquid form comprising pepstatin and magnesium alginate (composition according to the invention, Table 6).

Step 1:
Measure the water and pour it into the beaker with magnetic stirring;
Turn on the stirring and heat the water to 50° C.;
Add Buffer salts.

Raw materials: Demineralised water, di-sodium tetraborate, Boric acid.

Step 2:
Add the ingredients in the sequence reported (1. Sodium chloride, 2. Magnesium alginate) progressively waiting for complete dissolution;
Leave the obtained solution under stirring for 30 minutes.

Filtration step 1 The resulting solution should be filtered with a 0.2 μm PES filter.

Step 3:
After 1^{st} filtration, measure the water and pour it into a beaker, turn on the stirring and heat the water to 50° C.;
Add the ethanolic solution of pepstatin (for example, at a concentration of 1 mg/ml);
Leave the obtained solution under stirring for 30 minutes.

Pepstatin: Isovaleryl-L-val-L-val-statyl-L-alanyl-statin or Isovaleryl-L-val-L-val-4-(S)-amino-3-(S)-Hydroxy-6-methyl-heptanoyl-2-ala-4-(S)-amino-3-(S)-hydroxy-6-methyl-heptanoic acid).

Filtration step 2: The resulting solution should be filtered with a 0.2 μm PES filter.

TABLE 6
(final concentration in pepstatin about 1 μM)

| Components | amount | U.M. |
|---|---|---|
| PURIFIED WATER | 95.00-99.00 | g |
| DI-SODIUM TETRABORATE | 0.050-0.250 | g |
| BORIC ACID | 0.250-1.250 | g |
| MAGNESIUM ALGINATE | 0.050-0.350 | g |
| Na CHLORIDE ANHYDROUS | 0.200-0.760 | g |
| PEPSTATIN in EtOH 1 mg/mL | 0.005-0.100 | g |

Physical characteristics of the composition of Table 6:

| | |
|---|---|
| pH | about 7.53; |
| Osmolality | about 304 mOsm Kg⁻¹; |
| Viscosity | about 1.309 cSt; |
| Density | about 1.003 g mL⁻¹ |

Methods for measuring the reported physical quantities:

Osmolarity: osmometer

Viscosity: Ostwald viscometer (measured at room temperature)

pH: pH meter

Density: Pycnometer

Expected amount of pepstatin in the composition: less than or equal to 1μM.

Pepstatin soluble in ethanol up to 1 mg/mL.

Embodiments E(n) of the present invention are illustrated below:

E1. An aqueous composition for ophthalmic use comprising

(i) a mixture M comprising or, alternatively, consisting of:
(a) a pepstatin or a pharmaceutically acceptable salt thereof, and
(b) an alginic acid, or a pharmaceutically acceptable salt thereof; and

(ii) at least one ophthalmological grade additive and/or excipient.

E2. The composition according to E1, wherein said mixture M further comprises (c) hyaluronic acid or a pharmaceutically acceptable salt thereof.

E3. The composition according to E2, wherein said (i) mixture (M) comprises or, alternatively, consists of:

(a) a pepstatin;

(b) an alginate of an alkaline or alkaline-earth metal, preferably magnesium alginate; and,

(c) a hyaluronate of an alkaline or alkaline-earth metal, preferably a sodium hyaluronate.

E4. The composition according to any one of E1-E3, wherein the composition is formulated for ophthalmic use; preferably in liquid form for ophthalmic use, more preferably it is eye drops or aqueous-based eye drops; or, alternatively, in semi-solid form for ophthalmic use, preferably an ointment, a salve, a gel or a cream.

E5. The composition according to any one of E1-E4 for use as medicament.

E6. The composition according to any one of E1-E4 for use in a method for preventive and/or curative treatment of, a disease or symptom of the eyeball and/or periocular region related with or deriving from the presence of pepsin in the lacrimal fluid in a subject in need.

E7. The composition for use according to E6, wherein said disease or symptom of the eyeball and/or periocular region is selected from the group comprising or, alternatively, consisting of: lacrimal dysfunction syndrome (LDS) or dry eye syndrome, conjunctivitis of the conjunctiva, conjunctivitis of the cornea, ocular inflammation, periocular inflammation, blepharitis, keratitis, and uveitis; preferably selected from the group comprising or, alternatively, consisting of: lacrimal dysfunction syndrome (LDS) or dry eye syndrome, conjunctivitis of the conjunctiva, conjunctivitis of the cornea, ocular inflammation, and periocular inflammation.

E8. The composition for use according to E6 or E7, wherein said composition is for use in a method for the preventive and/or curative treatment of a disease or symptom of the eyeball and/or periocular region in: (i) a subject with a gastric reflux, which manifests itself from the stomach to an oesophageal and/or extra-oesophageal region, (ii) a subject with a disease or symptom related with or deriving from said gastric reflux.

E9. The composition for use according to E8, wherein said diseases or symptoms related with or deriving from, said gastric reflux, which manifests itself from the stomach to an oesophageal and/or extra-oesophageal region, are selected from the group comprising or, alternatively, consisting of: gastroesophageal reflux disease (GERD), erosive or non-erosive, laryngopharyngeal reflux (LPR) or extra-oesophageal reflux, oesophagitis, oesophageal ulcers, oesophageal mucosal de-epithelialisation, acid regurgitation, heartburn, feeling of gastric fullness, epigastric pain, dyspepsia, nausea, chronic cough, bronchospasm, sore throat, laryngitis, globus sensation or hypopharyngeal bolus, pyrosis, dysphonia, and rhinopharyngeal phlogosis.

E10. A process for the preparation of the composition according to any one of E1-E4, wherein said process comprises:

the step 1) for dissolving the pepstatin in a solvent, preferably an alcoholic solvent or ethanol, at neutral pH to obtain an alcoholic solution of pepstatin, followed by

the step 2) for diluting the alcoholic solution of pepstatin in an aqueous solution comprising an alginic acid salt, preferably an alginate of an alkaline or alkaline-earth metal, more preferably magnesium alginate.

1. An aqueous composition for ophthalmic use comprising
(i) a mixture M comprising or, alternatively, consisting of:
(a) a pepstatin or a pharmaceutically acceptable salt thereof, and
(b) an alginic acid, or a pharmaceutically acceptable salt thereof; and
(ii) at least one ophthalmological grade additive and/or excipient.

2. The composition according to claim 1, wherein said mixture M further comprises (c) hyaluronic acid or a pharmaceutically acceptable salt thereof.

3. The composition according to claim 2 wherein said (i) mixture (M) comprises or, alternatively, consists of:
(a) a pepstatin;
(b) an alginate of an alkaline or alkaline-earth metal, preferably a magnesium alginate; and,
(c) a hyaluronate of an alkaline or alkaline-earth metal, preferably a sodium hyaluronate.

4. The composition according to claim 1, wherein the composition is formulated for ophthalmic use; preferably in liquid form for ophthalmic use, more preferably it is eye drops or aqueous-based eye drops; or, alternatively, in semi-solid form for ophthalmic use, preferably an ointment, a salve, a gel or a cream.

5. The composition according to claim 1 for use as medicament.

6. The composition according to claim 1 for use in a method for preventive and/or curative treatment of a disease or symptom of the eyeball and/or periocular region related with or deriving from the presence of pepsin in the lacrimal fluid in a subject in need.

7. The composition for use according to claim 6, wherein said disease or symptom of the eyeball and/or periocular region is selected from the group comprising or, alternatively, consisting of:
lacrimal dysfunction syndrome (LDS) or dry eye syndrome, conjunctivitis of the conjunctiva, conjunctivitis of the cornea, ocular inflammation, periocular inflammation, blepharitis, keratitis, and uveitis; preferably selected from the group comprising or, alternatively, consisting of: lacrimal dysfunction syndrome (LDS) or dry eye syndrome, conjunctivitis of the conjunctiva, conjunctivitis of the cornea, ocular inflammation, and periocular inflammation.

8. The composition for use according to claim 6, wherein said composition is for use in a method for the preventive and/or curative treatment of a disease or symptom of the eyeball and/or periocular region in: (i) a subject with a gastric reflux, which manifests itself from the stomach to an oesophageal and/or extra-oesophageal region, (ii) a subject with a disease or symptom related with or deriving from said gastric reflux.

9. The composition for use according to claim 8, wherein said diseases or symptoms related with or deriving from, said gastric reflux, which manifests itself from the stomach to an oesophageal and/or extra-oesophageal region, are selected from the group comprising or, alternatively, consisting of:
gastroesophageal reflux disease (GERD), erosive or non-erosive, laryngopharyngeal reflux (LPR) or extra-oesophageal reflux, oesophagitis, oesophageal ulcers, oesophageal mucosal de-epithelialisation, acid regurgitation, heartburn, feeling of gastric fullness, epigastric pain, dyspepsia, nausea, chronic cough, bronchospasm, sore throat, laryngitis, globus sensation or hypopharyngeal bolus, pyrosis, dysphonia, and rhinopharyngeal phlogosis.

10. A process for the preparation of the composition according to claim 1, wherein said process comprises:
the step 1) for dissolving the pepstatin in a solvent, preferably an alcoholic solvent or ethanol, at neutral pH to obtain an alcoholic solution of pep statin, followed by
the step 2) for diluting the alcoholic solution of pepstatin in an aqueous solution comprising an alginic acid salt, preferably an alginate of an alkaline or alkaline-earth metal, more preferably magnesium alginate.

11. A method for treating a disease or symptom of the eyeball and/or periocular region related with or deriving from the presence of pepsin in the lacrimal fluid, comprising:
administering to the subject an aqueous composition comprising
(i) a mixture M comprising:
(a) a pepstatin or a pharmaceutically acceptable salt thereof, and
(b) an alginic acid, or a pharmaceutically acceptable salt thereof; and
(ii) at least one ophthalmological grade additive and/or excipient.

12. The method according to claim 11, wherein said mixture M further comprises (c) hyaluronic acid or a pharmaceutically acceptable salt thereof.

13. The method according to claim 12 wherein said (i) mixture (M) comprises:
(a) a pepstatin;
(b) magnesium alginate; and,
(c) sodium hyaluronate.

14. The method according to claim 11, wherein the composition is formulated as eye drops, or as an ointment, a salve, a gel or a cream.

15. The method according to claim 11, wherein said disease or symptom of the eyeball and/or periocular region is selected from the group consisting of: lacrimal dysfunction syndrome (LDS) or dry eye syndrome, conjunctivitis of the conjunctiva, conjunctivitis of the cornea, ocular inflammation, periocular inflammation, blepharitis, keratitis, and uveitis; preferably selected from the group comprising or, alternatively, consisting of: lacrimal dysfunction syndrome (LDS) or dry eye syndrome, conjunctivitis of the conjunctiva, conjunctivitis of the cornea, ocular inflammation, and periocular inflammation.

16. The method according to claim 15, wherein the subject has gastric reflux, which manifests itself from the stomach to an oesophageal and/or extra-oesophageal region.

17. The method according to claim 16, wherein said subject has gastroesophageal reflux disease (GERD), erosive or non-erosive, laryngopharyngeal reflux (LPR) or extra-oesophageal reflux, oesophagitis, oesophageal ulcers, oesophageal mucosal de-epithelialisation, acid regurgitation, heartburn, feeling of gastric fullness, epigastric pain, dyspepsia, nausea, chronic cough, bronchospasm, sore throat, laryngitis, globus sensation or hypopharyngeal bolus, pyrosis, dysphonia, or rhinopharyngeal phlogosis.
